(19) United States
(12) Patent Application Publication
(10) Pub. No.: US 2023/0103920 A1
(43) Pub. Date: Apr. 6, 2023

(54) DEVICE AND METHOD OF ANGIOGRAPHY FOR CEREBROVASCULAR OBLITERATION

(71) Applicant: Chung Yuan Christian University, Taoyuan City (TW)

(72) Inventors: Tzong-Rong Ger, Taoyuan City (TW); Chi-Ming Ku, Taoyuan City (TW); Chun-Hung Wang, Taoyuan City (TW)

(21) Appl. No.: 17/960,917

(22) Filed: Oct. 6, 2022

(30) Foreign Application Priority Data
Oct. 6, 2021 (TW) 110137182

(51) Int. Cl. A61B 6/00; G06V 10/24; G06V 10/44; G06V 10/26; G06T 5/20; G06T 5/00

(52) U.S. Cl. CPC A61B 6/5217; G06V 10/24; G06V 10/443; G06V 10/267; G06T 5/20; G06T 5/003; A61B 6/504; G06T 2207/30016; G06T 2207/10081; G06V 2201/031; G06T 2207/30008; G06T 2207/20192

(57) ABSTRACT

The present invention provides a method of angiography for cerebrovascular obliteration includes: using a classifier to obtain a 2D medical image using from a plurality of Multiphase CTA images; using a gray-scale conversion to obtain a N*M pixels grayscale image; filtering the grayscale image not being meet a condition of grayscale threshold and performing an image binarization to obtain a binarized image; confirming at least one vascular region and performing an image skeletonization; filtering according to a vascular image features of a vascular region to obtain a vascular-enhanced image; using a fracture analysis to obtain an analysis report related to a plurality of quantifying parameters of vascular characteristics; wherein the quantifying parameters of vascular characteristics comprises a quantitative value of fractal dimension (FD), vessel density (VD), skeleton density (SD) and vascular diameter index (VDI). Therefore, improve the accuracy of clinician diagnosis and the survival rates of patients with ischemic stroke

This application claims the benefit of Taiwan Patent Application Serial No. 110137182, filed Oct. 6, 2021, the subject matter of which is incorporated herein by reference.

### BACKGROUND OF INVENTION

### 1. Field of the Invention

The present invention is related to a technical field of medical image processing, and more particularly, to a computed tomography angiographic imaging method and imaging device that uses Fractal Geometry analysis to quantify the status of cerebral vascular collateral circulation for the assessment of ischemic stroke.

### 2. Description of the Prior Art

Among the top 10 causes of death by the World Health Organization in 2015, stroke ranked the second highest [1], indicating that the impact of stroke is not to be underestimated, and stroke can be divided into two types: hemorrhagic stroke caused by intracerebral hemorrhage and ischemic stroke caused by occlusion of blood vessels in the brain, among which ischemic stroke is the most common. Before ischemic stroke patients are treated, a clinician will conduct a preoperative assessment of the patient's condition to predict the postoperative clinical outcome to determine whether treatment should be performed.

Computed tomography angiography (CTA) has the advantages of short scanning time, easier access, and better observer agreement, and is a good imaging tool in the assessment of collateral circulation in patients with ischemic stroke. However, CTA is a monophasic imaging modality, and its lack of temporal information may lead clinicians to misjudge the status of collateral circulation. Therefore, multiphase computed tomography angiography (Multiphase CTA) has become a new option for clinicians to assess the status of collateral circulation. Circulation status assessment.

In the preoperative evaluation of patients with ischemic stroke, Multiphase CTA is a reliable tool for clinicians to assess the status of collateral circulation. However, nowadays, clinicians mostly rely on their rich clinical experience to evaluate the various stages of Multiphase CTA images. Therefore, it is important about a method or system that provides clinicians with reference imaging information and interpretation in the assessment of lateral branch circulation status.

### SUMMARY OF THE INVENTION

The present invention provides a method/system of angiography for cerebrovascular obliteration, and more particularly, to a computed tomography angiographic imaging method and imaging device that uses Fractal Geometry analysis to quantify the status of cerebral vascular collateral circulation for the assessment of ischemic stroke.

The Method mainly includes: using a classifier to obtain a 2D medical image using from a plurality of Multiphase CTA images; using a gray-scale conversion to obtain a N*M pixels grayscale image; filtering the grayscale image not being meet a condition of grayscale threshold and performing an image binarization to obtain a binarized image; confirming at least one vascular region and performing an image skeletonization; filtering according to a vascular image features of a vascular region to obtain a vascular-enhanced image; using a fracture analysis to obtain an analysis report related to a plurality of quantifying parameters of vascular characteristics; wherein the quantifying parameters of vascular characteristics comprises a quantitative value of fractal dimension (FD), vessel density (VD), skeleton density (SD) and vascular diameter index (VDI), and wherein the image skeletonization is performed by the Zhang skeletonization algorithm for image processing.

As a mentioned above, the invention mainly uses fragmentation analysis to quantify the information generated by the lateral branch circulation status, which provides clinicians with predictive clinical outcomes in the postoperative evaluation of patients suffering from ischemic stroke. The DICOM image format and the automatic correction of the brain images and the differentiation of the left and right hemispheres of the patient's brain into binarized vascular images and skeletonized images, which can calculate FD, VD, SD and VDI, can not only assist young physicians in the assessment of ischemic stroke, but also can quickly perform large data analysis, which can save physicians' time and physical effort, and further improve the accuracy of the clinician's diagnosis and the accuracy of the diagnosis. It can save time and effort for clinicians and improve the accuracy of diagnosis and survival rate of ischemic stroke patients.

In one embodiment, the fracture analysis is based on a box-counting dimension to generate a plurality of quantifying parameters of vascular characteristics.

In another embodiment, wherein the step of classifier to obtain a 2D medical image further comprises an image data filtering procedure, the image data filtering procedure includes: filtering the Multiphase CTA images that contain the 2D medical image at the base of the skull and the top of the skull.

In another embodiment, the method further comprises a normalization procedure and an edge enhancement procedure, the normalization procedure includes image centering, image angle correction, and left/right brain segmentation for the binarized image, and the edge enhancement procedure comprises background removal and re-binarization of the image after the normalization procedure.

### BRIEF DESCRIPTION OF THE DRAWINGS

The present invention will now be specified with reference to its preferred embodiment illustrated in the drawings, in which:

FIG. 1a illustrates a schematic diagram of the flow of the angiographic method described in this invention.

FIG. 1b illustrates a schematic diagram of a block of the angiographic device described in the invention.

FIGS. 2a and 2b are respectively graphical illustrations of the skull image removal operation described in the present invention.

FIGS. 3a-3c are respectively schematic diagrams of the normalized industry described in the present invention.

FIG. 4 shows the graphical illustration of the edge enhancement process described in the present invention.

FIG. 5 shows a schematic image of the vascular image feature filtering described in the present invention.

FIGS. 6a-6d show the enhanced images of blood vessels in the middle of the cerebral cavity of different patients described in this invention.

FIG. 8 is a schematic diagram of the operator interface in the angiographic device described in the present invention.

### DESCRIPTION OF THE PREFERRED EMBODIMENT

The invention disclosed herein is directed to a method/system of angiography for cerebrovascular obliteration. In the following description, numerous details are set forth in order to provide a thorough understanding of the present invention. It will be appreciated by one skilled in the art that variations of these specific details are possible while still achieving the results of the present invention. In other instance, well-known components are not described in detail in order not to unnecessarily obscure the present invention.

In order to have a clearer understanding of the technical features, purpose and effect of the present invention, the specific manner of implementation of the present invention is described in detail with respect to the attached drawings. However, the attached drawings are for reference and illustration purposes only and are not intended to limit the present invention; the foregoing and other technical contents, features and effects of the present invention will be clearly presented in the following detailed descriptions of each embodiment with reference to the drawings. The directional terms mentioned in the following examples, such as “up”, “down”, “left”, “right”, “front”, “back”, etc., are merely references to the directions shown in the additional illustrations. Therefore, the directional terms used are for illustrative purposes and are not intended to limit the present creation; furthermore, in each of the following embodiments, the same or similar components will be used with the same or similar component designations.

In order to have a clearer understanding of the technical features, purpose and effect of the present invention, the specific manner of implementation of the invention is described in detail with respect to the accompanying drawings. The terms “individual” and “patient” as used in connection with the present invention are used interchangeably to denote mammals including primates (e.g., monkeys, chimpanzees, and humans). In some embodiments, the individual or patient suffers from or is predisposed to a characteristic disease.

Furthermore, “one embodiment” or “an embodiment” or “in another embodiment” or “some embodiments” or “other embodiments” are referred to in this document. in another embodiment” or “some embodiments” or “other embodiments” means that the features, structures or characteristics of the specific object described in conjunction with the above embodiments are included in at least one embodiment. Accordingly, the phrases “in one embodiment” or “in an embodiment” or “in another embodiment” appear throughout this specification. “in another embodiment” or “in some embodiments” or “other embodiments” may not always mean the same embodiment. In addition, specific features, structures, or characteristics may be combined in one or more embodiments in any manner that is appropriate.

Before describing the present invention, the main motive of the present invention and the purpose and efficacy of developing the present invention based on this motive are mentioned. The angiographic method and the angiographic device described herein are based on the use of fragmentation analysis to quantify collateral circulation status to predict, evaluate, or verify postoperative clinical outcomes in patients with ischemic stroke. Accordingly, the present invention will be described later with respect to the application of collateral circulation status in predicting the outcome of ischemic stroke, the application of fractal analysis in medicine, and the application of fractal dimension in quantifying blood vessels.

In the preoperative evaluation of patients with ischemic stroke, Multiphase CTA is a reliable tool for clinicians to assess the status of collateral circulation. Fractal analysis is a methodology to quantify the filling rate and homogeneity of the target object, and has been used in many related applications in the quantitative analysis of medical images [8-11]. Fractal dimension (FD) and lacunarity (L) are the two most important parameters in fractal analysis to quantify the variation of lateral circulation in each phase of Multiphase CTA images. In collaboration with clinicians, we will obtain Multiphase CTA images of patients with ischemic stroke and the region of interest (ROI) selected by clinicians, and perform pre-image processing on the CTA images to extract vascular information, and then perform fragmentation analysis to quantify the changes in collateral circulation for clinicians in Finally, the information is used to construct a deep learning model to establish a reliable computer assisted techniques (CAT).

Based on the application of collateral circulation in predicting the outcome of ischemic stroke; in 2011, Oh Young Bang et al. evaluated the relationship between preoperative collateral circulation status and revascularization outcome after endovascular treatment in 138 US and 84 Korean patients with ischemic stroke from two different groups. The results of the study showed that only 14.1% of patients with poor collateral circulation had complete revascularization, while 25.2% of patients with good collateral circulation and 41.5% of patients with excellent collateral circulation had complete revascularization (P<0.001).

Based on the application of fragmentation analysis in medicine; the tumor vessels of hepatocellular carcinoma have focal leakage and uneven blood flow, therefore, anti-vascular therapy is chosen, however, nowadays, the assessment tool for treatment effect is mainly using Response Evaluation Criteria in Solid Tumors (RECIST). However, the main tool for evaluating the therapeutic effect is the Response Evaluation Criteria in Solid Tumors (RECIST), which is an invasive test, so Koichi Hayano et al. proposed in 2014 to use the fragmentation dimension of computed tomography perfusion images (CTP images) to evaluate the therapeutic effect of HCC. The effect of HCC treatment was evaluated using the fractal dimension of CTP images. They used Image J's fractal analysis plug-in Fraclac to perform fractal analysis, selected the box-counting algorithm, and used Progression-free Survival (PFS) as the threshold for six months to differentiate the treatment effect. The results of the study showed that clinical outcome was significantly correlated with the change in the fragmentation dimension of CTP images before and after treatment (P=0.01), and the fragmentation dimension of CTP images generally decreased after treatment (PFS≥6 months), indicating that the tumor vascularity was significantly suppressed.

However, Neuroglioma is a heterogeneous tumor of neuroglial cells, which can be classified into different grades, Therefore, Smitha, K A et al. in 2015 proposed to use the two most important parameters of fractal analysis, Fractal dimension (FD) and Lacunarity (L), to analyze MRI images to evaluate the grade of glioma. The results of this study showed that both fractal dimension and interstitial dimension were significantly different in the comparison of high and low grade gliomas, with interstitial dimension performing better (P(FD)=0.04, P(L)=0.001), demonstrating that both fractal dimension and interstitial dimension can be used as reference standards for differentiating glioma grades in MRI fractal analysis.

As mentioned above, the angiographic method and angiographic device described herein are based on the use of fragmentation analysis to quantify the status of collateral circulation to predict, evaluate, or verify the postoperative clinical outcome of a patient with an ischemic stroke. The theoretical model of the invention by means of fractal analysis is described below.

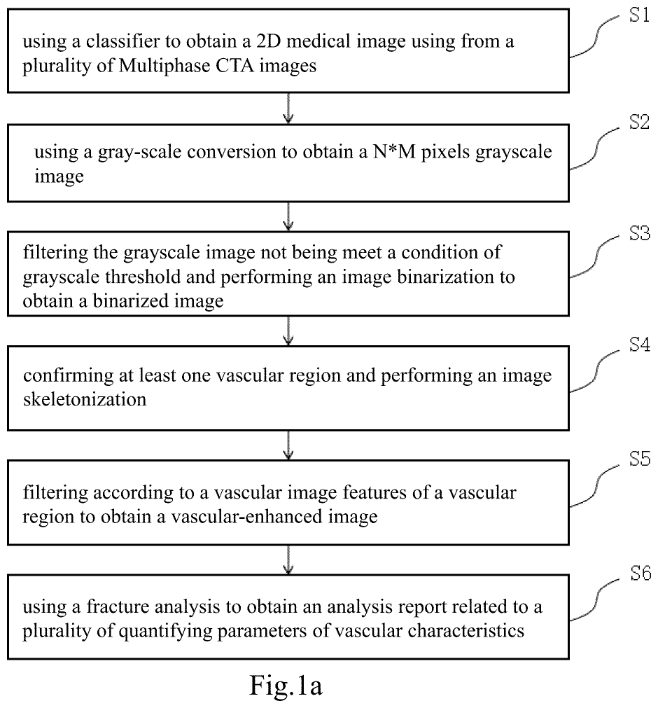

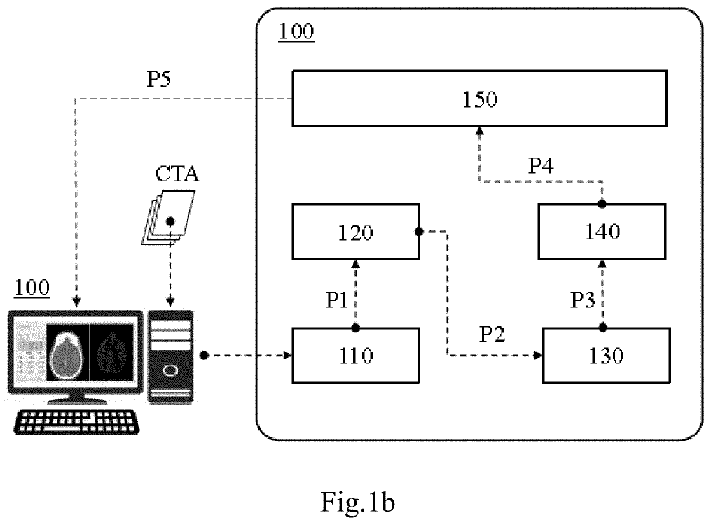

Referring to FIG. 1a, it shows a flowchart illustrating the angiographic method described in the present invention. The angiographic method comprises steps of:

Step 1: using a classifier to obtain a 2D medical image using from a plurality of Multiphase CTA images;

Step 2: using a gray-scale conversion to obtain a N*M pixels grayscale image;

Step 3: filtering the grayscale image not being meet a condition of grayscale threshold and performing an image binarization to obtain a binarized image;

Step 4: confirming at least one vascular region and performing an image skeletonization;

Step 5: filtering according to a vascular image features of a vascular region (ROI) to obtain a vascular-enhanced image; and

Step 6: using a fracture analysis to obtain an analysis report related to a plurality of quantifying parameters of vascular characteristics.

According to the above method, wherein the vascular image features include selected image grayscale values, gradient values, contrast values, shape contours, grayscale value variance, position relationships, or a combination of any of the above, and wherein the quantifying parameters of vascular characteristics comprises a quantitative value of fractal dimension (FD), vessel density (VD), skeleton density (SD) and vascular diameter index (VDI).

Referring to FIG. 1b, the present invention further provides a system of angiography for cerebrovascular obliteration, comprises: a computer 100, which includes: an image filtering module 110, a conversion module 120, a binarization module 130, a feature filtering module 140 and a fragmentation analysis module 150; wherein the image filtering module 110 is configured to filter a plurality of Multiphase CTA images to form a 2D medical image; the conversion module 120 is configured to perform a gray-scale conversion to form a N*M pixels grayscale image; the binarization module 130 is configured to filter the grayscale image not being meet a condition of grayscale threshold and perform an image binarization to form a binarized image; the feature filtering module 140 is configured to confirm at least one vascular region, perform an image skeletonization and filter according to a vascular image features of a vascular region to form a vascular-enhanced image; and the fragmentation analysis module 150 is configured to using a fracture analysis for the vascular-enhanced image to form an analysis report related to a plurality of quantifying parameters of vascular characteristics;

According to the above system, wherein the vascular image features include selected image grayscale values, gradient values, contrast values, shape contours, grayscale value variance, position relationships, or a combination of any of the above, and wherein the quantifying parameters of vascular characteristics comprises a quantitative value of fractal dimension (FD), vessel density (VD), skeleton density (SD) and vascular diameter index (VDI).

The fractal dimension is a morphology that quantifies the complexity of an object, defined as a fractal shape that can be divided into several small objects that have similar properties to the original shape. For complex objects that cannot be represented by integration, Benoit Mandelbrot introduced the theory of fractional shapes. Theoretically, the smaller the scale of the fractal, the more accurate the complexity of the object can be measured, and the fractal scale has a linear relationship with the complexity on a logarithmic graph. Fractals can be used to measure the complexity of length, area, or volume, and FD can describe many natural geometric features, such as self-similarity in the texture or structure of a living object.

In nature, not all objects conform to the perfect fragmentation feature, and it is difficult to find the self-similarity of almost all objects in nature. The Box-counting algorithm first defines the Box size, puts the fragmented pattern into the Box, and then calculates the total number of Boxes occupied by the fragmented pattern. The slope of the exponential graph is the fragmentation dimension. Under the box counting algorithm, unlike the fractal dimension, the gap degree is calculated as the change in the number of pixels occupied by each measure square, rather than the counted number of measure squares, where the gap degree (L) can be expressed as below:

L=(σε/με)²

wherein σε is the standard deviation of the foreground pixels in the scale square when the multiplicity of the current scale square to the background square is ε; με is the average value of the foreground pixels in the scale square when the multiplicity of the current scale square to the background square is ε.

In ImageJ, the calculation of the gap degree also takes into account the grid orientation, so the formula for calculating the gap degree (L) in ImageJ can be expressed as below:

L=(Σ_{g=1}^{Grid positions}Λg)/Grid positions

Λg=(Σ_{ε=min box size}^{max box size}(σεg/μεg)²)/N

wherein Λg is the gap when the grid orientation is g; σεg is the standard deviation of foreground pixels in the scale square when the grid orientation is g and the current scale square to background square multiplicity is ε; μεg is the average of foreground pixels in the scale square when the grid orientation is g and the current scale square to background square multiplicity is ε; and N is the multiplicity of several scale squares to background square used.

The following ImageJ software image processing involves Gaussian filtering, FFT, color image edge extraction, mathematical morphology and image fusion methods to extract vascular information; the fractal analysis of cerebral vascular information is performed by using the two most important parameters of fractal analysis, fractal dimension and interval, to quantify the variation of vessels in each phase of Multiphase CTA. The parameters quantified by the fractal analysis will be identified by statistical analysis and receiver operating characteristic curve (ROC curve), and finally, the identification results of the quantified parameters will be used to construct a deep learning model to establish A reliable evaluation method for stroke angiography will be established.

As mentioned above, the image processing of Multiphase CTA images is broadly divided into the following stages. Before the image processing, a pre-image processing is first performed, which includes an image data conversion process and an image data filtering process. The DICOM file will be adjusted to a fixed brightness range (−225˜525) and converted to 8-bit grayscale image (grayscale value of 0˜255), and then convert it to a common image file format for subsequent image pre-processing. The image data filtering process is mainly due to the fact that the physiologically significant Multiphase CTA images near the base of the skull and the top of the skull have too many bones to easily observe the trend of cerebrovascular changes. Therefore, the image data filtering process is performed before the first stage, which filters the medical image data containing the medical image data at the base of the skull and the top of the skull.

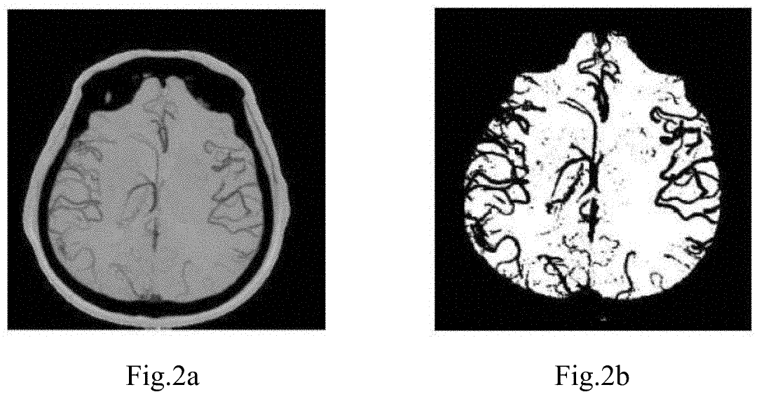

The first stage: Remove skull image operation, as shown in FIGS. 2a and 2b: mainly includes the process of removing cavity and skull stripping; wherein the removal of cavities is performed manually by the polygon selections function of Image J, and the clear outside function is used to remove the information outside the circled area to remove the white cavities from the grayscale image, i.e., whether the above-mentioned parts satisfy the specific grayscale value condition. Reverse the grayscale; for example, convert the part of the grayscale image with pixel grayscale value greater than 10 (including 10) to 0 (black), and keep the other parts with the original grayscale value to obtain the grayscale image after removing the cavity; and the skull stripping is to differentiate the cerebral blood vessels and their information in the brain in a unified way, and to binarize the grayscale image after removing the cavity. In this example, the invention mainly uses the grayscale value of 128 as the threshold value, converts the grayscale value below 128 to grayscale value 0 (black), and the grayscale value above 128 to grayscale value 255 (white), and then performs Canny edge detection on the binarized image to obtain the skull edge, and transforms the pixel value outside the skull edge to 0 to obtain the binarized image after skull debridement.

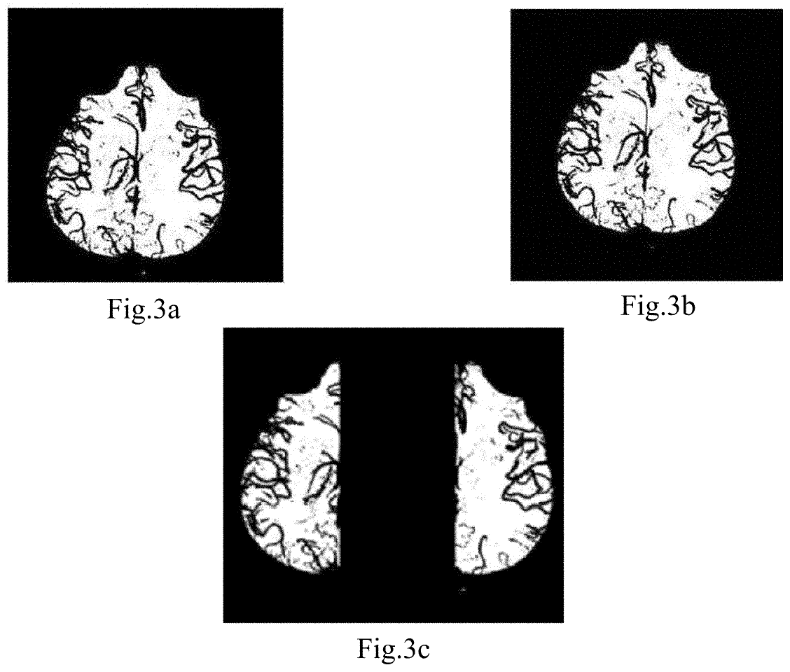

The second stage: the normalization procedure, as shown in FIGS. 3a-3c: The normalization operation mainly includes image centering and left/right brain image segmentation processing procedures, because the ten brains of the patient's CTA images are difficult to be in the middle position, and the subsequent left/right brain image segmentation cannot be smoothly divided, so the image centering processing procedure must be carried out first to move the brain image to the center. The main purpose is to detect the location of the upper/lower/left/right border to calculate the center of the brain, and then adjust the whole image to make it centered according to the center of the brain, and then proceed to the processing of the left/right brain image segmentation to obtain the cut image of the left and right brain after segmentation, and then proceed to the third stage.

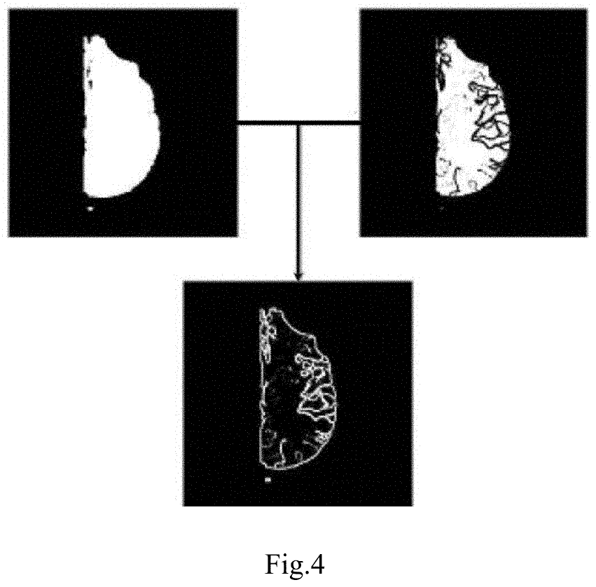

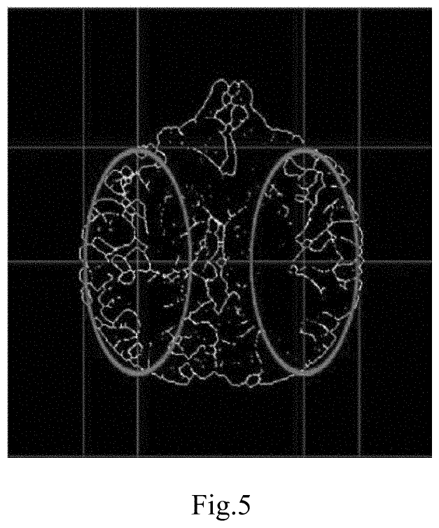

The third stage: Edge enhancement process, as shown in FIG. 4: After removing the skull, the overall image background is white, and the shatter analysis must make the analysis target as foreground pixels, so the white background should be removed. In this example, the Rolling ball radius is 50 pixels, but it is not limited to that. In order to eliminate the junction between the brain and the skull after removing the background, the grayscale value of the junction will be reduced after the background removal function, and the image will be binarized again.

The fourth stage: Image Skeletonization: Since the target of the analysis is the distribution of blood vessels in the brain and does not include blood flow and other factors, the vascular information will be skeletonized and the image analysis software Image J's Skeletonize function will be used for skeletonization processing to extract enhanced images of blood vessels to complete the pre-image processing operation. In this embodiment, in order to obtain the skeletonized image that can later compute the image parameters of blood vessels, the skeletonization algorithm described in this invention uses the Zhang algorithm, which is mainly divided into two steps. N(p1) means the number of 8 pixels adjacent to p1 that are not 0 at the end, S(p1) means the number of times the pixel value changes from 0 to 1 between p2, p3 . . . p9 and finally p2 in order, and p1 is deleted when the four conditions of each step are satisfied.

| Step. 1 | Step. 2 | | | |
|---|---|---|---|---|
| (1) 2 ≤ N(p1) ≤ 6 | (1) 2 ≤ N(p1) ≤ 6 | p9 | p2 | p3 |
| (2) S(p1) = 1 | (2) S(p1) = 1 | p8 | p1 | p4 |
| (3) p2 · p4 · p6 | (3) p2 · p4 · p8 | p7 | p6 | p5 |
| (4) p4 · p6 · p8 | (4) p2 · p6 · p8 | | | |

The fifth stage: Determine the vascular region (ROI) and extract the vascular enhancement image, as shown in FIG. 5: After the above image binarization and skeletonization process, the vascular enhancement image is obtained, and the length from the center of the lateral circulation vessels in the left/right brain to the border of the skull is calculated by the program, and the area of the lateral circulation vessels in the left/right brain is selected (ellipse area in the figure) for the subsequent calculation of the quantitative parameters of the vascular features in the ellipse area.

The sixth stage: The enhanced images of blood vessels extracted after completing the above pre-image processing will be fragmented to generate one or more vascular characteristic analysis samples with quantitative parameters. The fragmentation plug-in Fraclac of image analysis software ImageJ is used to perform the fragmentation analysis of the vascular information and quantify the changes of the vessels at each stage to generate one or more vascular characteristics quantification parameters of the vascular characteristics analysis samples. In this embodiment, the box-counting algorithm is used to analyze the fractal shape and obtain the fractal dimension (FDB) and gap information; the parameters of the box-counting algorithm are set as follows: the minimum grid size is 5*5 pixels to reduce the error caused by the image pixel limitation; the maximum grid size is 45% of the background grid to avoid the meaningful oversize of the grid, and the grid positions are set as 12 to reduce the box-counting deviation caused by the grid direction.

As mentioned above, the quantitative parameters of vascular characteristics include Vessel Density (VD), Skeleton Density (SD), Vascular Diameter Index (VDI), and Fractal dimension (FD). Vascular Diameter Index (VDI), Fractal dimension (FD). Vascular density (VD) is defined as the ratio of the total area of all images to the area occupied by the vessels in the binarized image. Assuming that in an image of length and width n×n, where n is the length of the image, B(i,j) represents the white pixels in the pre-skeletonization image, which also represents the portion of blood vessels in the pre-skeletonization image of CTA, and X(i,j) represents the is the pixels of all images, (i,j) represents the coordinates of the images, and VD is the Vessel Density (VD).

VD=ΣB(i,j)n(i,j)(ΣX(i,j)n(i,j))2

Vascular skeletal density (SD) is defined as the ratio of the image area to the area of the skeletal part of all images by the following equation, and the fragmentation dimension will also be calculated from the skeletonized images. The vascular skeletal density (SD) is similar to the vascular density (VD) in images of length and width n×n, where n is the length of the image, L(i,j) represents the white pixels in the image after skeletonization, which also represents the portion of the vascular skeleton in the CTA image, and X(i, X(i,j) represents the pixels of all images, (i,j) represents the coordinates of the images, and SD is the Skeleton Density (SD).

SD=ΣL(i,j)n(i,j)(ΣX(i,j)n(i,j))2

Vessel diameter index (VDI) is calculated using vascular density (VD) and vascular skeletal density (SD). Vessel diameter index (VDI) is defined as the ratio of vascular density (VD) to vascular skeletal density (SD). In an image of length and width n×n, B(i,j) represents the portion of blood vessels in the CTA image, L(i,j) represents the portion of the vessel skeleton in the CTA image, and VDI is the Vessel Diameter Index (VDI). Index).

VDI=VDSD=ΣB(i,j)n(i,j)ΣL(i,j)n(i,j)

The FD value is calculated by the slope of the logarithm of the Box size and the number of Boxes, and there is no perfect fractal pattern in nature. When calculating the FD for different types of objects, the slope of the FD for each box size should be recorded first, and the FD with the higher linearity should be selected instead of calculating the slope of the whole FD.

As mentioned above, the basic definition of vascular density (VD) is the density of blood vessels in a CTA image, and the white pixels represent blood vessels in the physiological sense after the above image processing. In other words, to calculate the density of blood vessels, the ratio of the number of white pixels in the image to the number of pixels in the whole image should be calculated.

The difference between fractal dimension (FD) and vascular skeletal density (SD) versus vascular density (VD) is the choice of image source. Vascular density (VD) uses images of the vessel before skeletonization, which contains information about the length and width of the vessel. Vascular skeletal density (SD) and fractal dimension (FD) use skeletal images. The physiological significance of vascular skeletal density (SD) represents vessel length information, and no additional information is needed for the calculation, so skeletal images are used to remove vessel width information Similar to the vascular skeletal density (SD), the fractured dimension (FD) represents the shape dimension. The original pre-skeleton vascular images may contain small hairy edges at the vascular borders, which may affect the value of the fractured dimension (FD) in the calculation of the shape dimension, and therefore the skeletalized images must be used for image selection. Finally, the vessel diameter index (VDI) represents the width of the vessel. To extract the width information from the VDI, the length information of the VDI is removed from the VDI for calculation, which is the skeletal density (SD). Vessel width information (VDI) was obtained by dividing VD and SD.

Based on the information from the above image processing and fragmentation analysis, the present invention includes an operator interface for the angiographic device based on the evaluation of brain stroke, as shown in FIG. 8; wherein the operator interface can be divided into four functional areas, (1) interface operation area, (2) image display area, (3) parameter display area and (4) save and close. The interface operation area can read all DICOM files in the folder, and sort and display DICOM images automatically. The image display area displays the original DICOM images and the processed images for users to view. The parameter display area shows the vascular parameters of the left and right brain. The final save and close can store DICOM images and vascular parameters for users to record patient clinical information.

The image display area in FIG. 8 contains two images, the left image is the original CTA image and the right image is the image after image processing. At this time, all images in the DICOM folder can be displayed. After “Image analysis”, the original CTA images will be filtered to show five images with physiological significance of stroke.

In the leftmost parameter display area, there are eight parameters, FD, VD, SD, VDI for the left brain and FD, VD, SD, VDI for the right brain. The vascular parameters of the left and right brains displayed in this area are obtained from the calculation and analysis of the patient's brain based on the CTA images taken, rather than the visual direction of the left and right side of the user's vision, because the direction of the CTA images taken is opposite to the visual direction, so for the convenience of the user, the parameters of the left brain are placed in the right direction when displayed. Conversely, the right brain parameters were placed in the left direction. In addition, users who want to record DICOM images and vascular parameters can save the values of five physiologically significant CTA images and vascular parameters in the current DICOM folder.

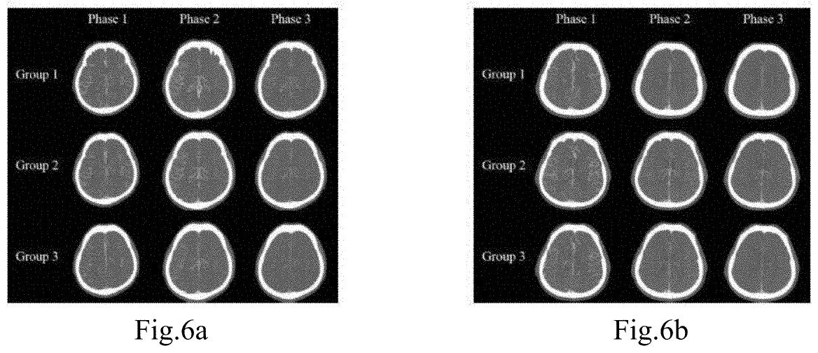

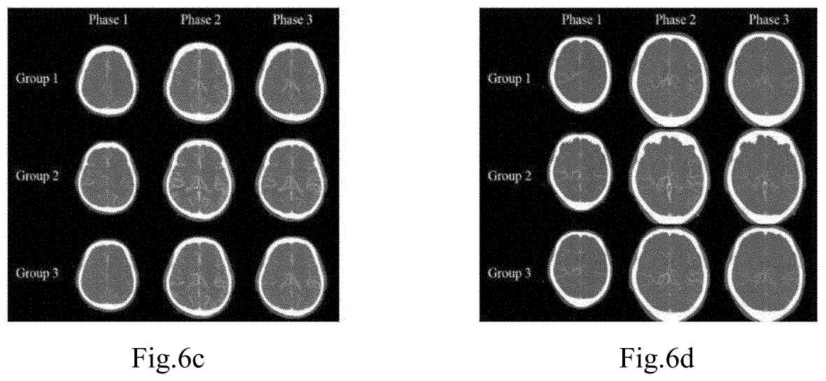

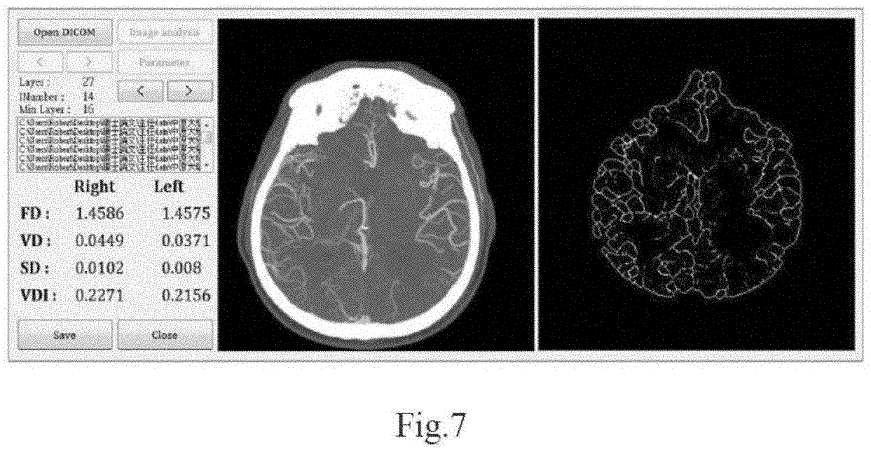

According to the angiographic method and angiographic device described above, the present invention selects three groups of vascular enhancement images, such as case1, case2, case3 and case4 of FIGS. 6a-6d, from the middle of the brain cavity of four patients after pre-processing of the images, and the ROI area is circled through the cooperation with clinicians. In addition, another objective of this invention is to predict the postoperative clinical outcome of ischemic stroke, and the clinical outcome is determined using the Modified Rankin Scale (mRS), which is the most widely used clinical outcome indicator. The mRS scale scores are shown in Table 1 below. Cases with mRS scale scores less than 3 were considered favorable, while cases with mRS scale scores greater than or equal to 3 were considered unfavorable clinical outcomes.

| Score | Description |
|---|---|
| 0 | No symptoms |
| 1 | No significant disability. Able to carry out all usual activities, |
| | despite some symptoms |
| 2 | Slight disability. Able to look after own affairs without |
| | assistance, but unable to carry out all previous activities |
| 3 | Moderate disability. Requires some help, but able to walk |
| | unassisted |
| 4 | Moderately severe disability. Unable to attend to own bodily |
| | needs without assistance, and unable to walk unassisted |
| 5 | Severe disability. Requires constant nursing care and attention, |
| | bedridden, incontinent |
| 6 | Dead |

The vascular filling rate and complexity of the collateral circulation were quantified by the fractal dimension (FDB), and the collateral circulation scoring criteria of Multiphase CTA were determined in part by the changes in the collateral circulation of each phase, as shown in Table 2 below.

| Score | Description |
|---|---|
| 0 | When compared with the asymptomatic contralateral hemisphere, |
| | there are no vessels visible in any phase within the ischemic |
| | vascular territory |
| 1 | When compared with the asymptomatic contralateral hemisphere, |
| | there are just a few vessels visible in any phase within the |
| | occluded vascular territory |
| 2 | When compared with the asymptomatic contralateral hemisphere, |
| | there is a delay of two phases in filling in of peripheral vessels |
| | and decreased prominence and extent or a one-phase delay and |
| | some ischemic regions with no vessels |
| 3 | When compared with the asymptomatic contralateral hemisphere, |
| | there is a delay of two phases in filling in of peripheral vessels |
| | or there is a one-phase delay and significantly reduced number |
| | of vessels in the ischemic territory |
| 4 | When compared with the asymptomatic contralateral hemisphere, |
| | there is a delay of one phase in filling in of peripheral vessels, |
| | but prominence and extent is the same |
| 5 | When compared with the asymptomatic contralateral hemisphere, |
| | there is no delay and normal or increased prominence of pial |
| | vessels/normal extent within the ischemic territory in the |
| | symptomatic hemisphere |

According to the above embodiments, the present invention further proposes a possible implementation of a risk identification method for evaluating brain stroke based on the information generated by the above angiographic method and angiographic device, which risk identification method broadly comprises the following steps:

Step A: providing at least one of the quantifying parameters of vascular characteristics to compare with a reference data, to obtain a normalized comparison data (complete ratio of collateral); and

Step B: using the normalized comparison data to compare a Modified Rankin-Scale (mRS) to obtain a level grading result.

Step C: reading the level grading result as a risk assessment indicator for assessing stroke and treatment outcome prediction; wherein the level grading result is based on a scale of 0 to 6, and the scale of 0 to 3 is being as a first appraisal indication, and the scale of 4 to 6 is being as a second appraisal indication.

According to an embodiment of the present invention, wherein the normalized comparison data is used to statistically identify parameters, which comprises SPSS or Random Forest, and determine the predictive model for those parameters and then the characteristic curve (ROC curve) is operated to verify the area, sensitivity and specificity.

In conclusion, the present invention reveals an angiographic method and an angiographic device based on the evaluation of cerebral stroke, which can quantify the cerebral vascular collateral circulation in Multiphase CTA images by four vascular characteristic quantification parameters, FD, VD, SD and VDI, and design a method that can automatically quantify the cranial collateral vascular parameters by automatically quantifying the cranial collateral vascular parameters. Digital Imaging and Communications in Medicine (DICOM) can be directly read and automatically correct the brain images and differentiate the left and right half of the patient's brain images, and process the left and right half of the brain images into binarized vascular images and skeletonized images, which can calculate FD, VD, SD and VDI for physicians' reference to analyze vascular parameters.

Therefore, This invention not only assists young physicians in the assessment of ischemic stroke, but also enables rapid analysis of large data, which saves physicians' time and effort, thereby improving the accuracy of diagnosis and survival of ischemic stroke patients by clinicians.

While the present invention has been particularly shown and described with reference to a preferred embodiment, it will be understood by those skilled in the art that various changes in form and detail may be without departing from the spirit and scope of the present invention.

What is claimed is:

1. A method of angiography for cerebrovascular obliteration, comprising:
using a classifier to obtain a 2D medical image using from a plurality of Multiphase CTA images;
using a gray-scale conversion to obtain a N*M pixels grayscale image;
filtering the grayscale image not being meet a condition of grayscale threshold and performing an image binarization to obtain a binarized image;
confirming at least one vascular region and performing an image skeletonization;
filtering according to a vascular image features of a vascular region to obtain a vascular-enhanced image; and
using a fracture analysis to obtain an analysis report related to a plurality of quantifying parameters of vascular characteristics;
wherein the quantifying parameters of vascular characteristics comprises a quantitative value of fractal dimension (FD), vessel density (VD), skeleton density (SD) and vascular diameter index (VDI).

2. The method of claim 1, wherein the fracture analysis is based on a box-counting dimension to generate a plurality of quantifying parameters of vascular characteristics.

3. The system of claim 1, wherein the step of classifier to obtain a 2D medical image further comprises an image data filtering procedure, the image data filtering procedure includes:
filtering the Multiphase CTA images that contain the 2D medical image at the base of the skull and the top of the skull.

4. The method of claim 1, wherein further comprises a normalization procedure, which includes image centering, image angle correction, and left/right brain segmentation for the binarized image.

5. The method of claim 4, wherein further comprises an edge enhancement procedure, which includes background removal and re-binarization of the image after the normalization procedure.

6. The method of claim 1, wherein the image skeletonization is performed by the Zhang skeletonization algorithm for image processing.

7. The method of claim 1, wherein the vascular image features is selected from an image grayscale value, a gradient value, a contrast value, a shape contour, a grayscale value variance, a position relationship, or a combination of anyone.

8. The method of claim 1, wherein the condition of grayscale threshold is 128 (the grayscale value), the binarized image is obtained after filtering the grayscale image, which includes:
converting grayscale values below 128 to 0;
converting grayscale values over 128 to 255; and
performing Canny edge detection on the binarized image to obtain the image of skull edge and convert the pixel value outside the skull edge to 0.

9. The method of claim 1, further comprising a risk evaluation which includes:
providing at least one of the quantifying parameters of vascular characteristics to compare with a reference data, to obtain a normalized comparison data; and
using the normalized comparison data to compare a Modified Rankin—Scale to obtain a level grading result;
wherein the level grading result is used for risk evaluation of stroke and treatment outcome prediction.

10. The method of claim 9, wherein the normalized comparison data is used to statistically identify parameters, which comprises SPSS or Random Forest, and determine the predictive model for those parameters.

11. The method of claim 9, wherein the level grading result is based on a scale of 0 to 6, and the scale of 0 to 3 is being as a first appraisal indication, and the scale of 4 to 6 is being as a second appraisal indication.

12. A system of angiography for cerebrovascular obliteration, comprising a computer, which includes: an image filtering module, a conversion module, a binarization module, a feature filtering module and a fragmentation analysis module;
the image filtering module is configured to filter a plurality of Multiphase CTA images to form a 2D medical image;
the conversion module is configured to perform a gray-scale conversion to form a N*M pixels grayscale image;
the binarization module is configured to filter the grayscale image not being meet a condition of grayscale threshold and perform an image binarization to form a binarized image;
the feature filtering module is configured to confirm at least one vascular region, perform an image skeletonization and filter according to a vascular image features of a vascular region to form a vascular-enhanced image; and
the fragmentation analysis module is configured to using a fracture analysis for the vascular-enhanced image to form an analysis report related to a plurality of quantifying parameters of vascular characteristics;
wherein the quantifying parameters of vascular characteristics comprises a quantitative value of fractal dimension (FD), vessel density (VD), skeleton density (SD) and vascular diameter index (VDI).

13. The system of claim 12, wherein the fracture analysis is based on a box-counting dimension to generate a plurality of quantifying parameters of vascular characteristics.

14. The system of claim 12, wherein the feature filtering module further is configured to filter the Multiphase CTA images that contain the 2D medical image at the base of the skull and the top of the skull.

15. The system of claim 12, wherein the feature filtering module further is configured to perform a normalization procedure, which includes image centering, image angle correction, and left/right brain segmentation for the binarized image before performing the image skeletonization.

16. The system of claim 15, wherein the feature filtering module further is configured to perform an edge enhancement procedure, which includes background removal and re-binarization of the image after performing the normalization procedure.

17. The system of claim 12, wherein the image skeletonization is performed by the Zhang skeletonization algorithm for image processing.

18. The system of claim 12, wherein the vascular image features is selected from an image grayscale value, a gradient value, a contrast value, a shape contour, a grayscale value variance, a position relationship, or a combination of anyone.
